(19) United States
(12) Patent Application Publication
(10) Pub. No.: US 2002/0065464 A1
(43) Pub. Date: May 30, 2002

(54) Imaging device

(76) Inventors: Kieran P Murphy, Baltimore, MD (US); T. Andrew Currier, Toronto (CA)

Correspondence Address: Kieran P Murphy, 119 Beechdale Road, Baltimore, MD, 21210, US

(21) Appl. No.: 09/726,130

(22) Filed: Nov. 30, 2000

(51) Int. Cl. A61B 8/00

(52) U.S. Cl. 600/437

(57) ABSTRACT

The present invention provides a novel system and method for capturing medical images. In an aspect of the invention, there is provided a wireless mobile unit that houses an ultrasonic transducer and is operable to capture ultrasonic images from a patient, and wirelessly transmit those captured images to a processing unit. The processing unit is operable to receive the images and present those images on an output device, such as a monitor.

### FIELD OF THE INVENTION

The present invention relates generally to medical imaging and more particularly relates to medical imaging devices.

### BACKGROUND OF THE INVENTION

Medical imaging technology is well known. X-rays, Magnetic Resonance Imaging (MRI) and computerized axial tomography (CT) are all fairly mature imaging technologies. Another mature imaging technology is ultrasound, which is often used for obtaining image of the fetus during pregnancy—it is considered particular suitable for this task (in contrast to X-rays) because the ultrasound waves which are emitted into the patient's body are mechanical in nature, and therefore less likely to interfere with the fetus.

One problem, however, with modem ultrasound machines is that they tend to require a large processing unit, often the size of a refrigerator, which connects to a handheld transducer by a cable, which on some models can be up to one inch thick. This configuration limits access in confined spaces, and the operator of the machine must be within arms-length of the processing unit in order to adjust the controls. One currently, popular type of ultrasound machine is the PowerVision™ 6000edp offered by Toshiba America Medical Systems, Inc. of 2441 Michelle Drive, PO Box 2068, Tustin Calif.

Some of the foregoing disadvantages are obviated by portable ultrasound machines, such as the SonoSite™ 180 offered by Sonosite Inc., 19807 North Creek Parkway, Suite 200, Bothwell, Wash. The SonoSite 180 includes a small, portable, hand-carried processing unit that is connected to a transducer. Unfortunately, this still requires the operator to interact with both the transducer and the processing unit while performing the ultrasound. Furthermore, the SonoSite 180's processing unit lacks some of the functionality of the larger machines, such as the PowerVision 6000edp. In general, the SonoSite 180 does not offer more complex image processing functions available in certain larger machines.

It is known to provide certain types of ultrasound processing units which connect to their transducer via a wireless link, as taught in U.S. Pat. No. 5,865,733 to Malinouskas, et al. However, Malinouskas is principally directed to the monitoring of physiological functions using a wireless optical transducer which is attached to the patient. Malinouskas merely teaches an alternative embodiment of a wireless ultrasound transducer for monitoring physiological functions, and such a device is not readily suitable for, or adaptable for use in medical imaging operations. Malinouskas also uses non-imaging pulsed Doppler to identify the Doppler shift and hence record and measure blood flow, and thus has no imaging capability, but merely transmits numerical data only. Malinouskas does not teach transmission of images of tissue, either normal or abnormal, nor measure depth, width, identify location, or identify pathological structures.

### SUMMARY OF THE INVENTION

Accordingly, it is an object of the invention to provide a novel imaging device that obviates or mitigates at least one of the disadvantages of the prior art.

In an aspect of the invention there is provided a system for gathering images comprising a mobile unit having a transducer, a microprocessor, a modem and a radio for capturing or gathering images and transmitting the images over a wireless link. The system also includes and a processing unit having a radio, a modem, a microprocessor and an output device for receiving, via the wireless link, the transmitted images and presenting the images on the output device. The output device can be any suitable device, such as a monitor or printer.

In another aspect of the invention, there is provided method for gathering or capturing images comprising the steps of:

capturing an analog image representation of at least a portion of a patient;

converting the analog image representation into a digital representation; and, compressing the image;

modulating the image into a format for wireless transmission;

transmitting the modulated image over a wireless link.

receiving the transmitted image at a processing unit;

demodulating the received image; and,

presenting the image on an output device.

In another aspect of the invention, there is provided a mobile unit for capturing images and for use in conjunction with a processing unit having a radio, a modem, a microprocessor and an output device for receiving, via a wireless link, the images and presenting the images on the output device, the mobile unit comprising: a transducer, a microprocessor, a modem and a radio for gathering images and transmitting the images over the wireless link to the processing unit.

In another aspect of the invention, there is provided a processing unit for receiving from captured images from a mobile unit having a transducer, a microprocessor, a modem and a radio and for transmitting the images over a wireless link, the processing unit comprising: a radio, a modem, a microprocessor and an output device for receiving, via the wireless link, the transmitted images and presenting the images on the output device.

The present invention provides a novel system and method for capturing medical images. In an aspect of the invention, there is provided a wireless mobile unit that houses an ultrasonic transducer and is operable to capture ultrasonic images from a patient, and wirelessly transmit those captured images to a processing unit. The processing unit is operable to receive the images and present those images on an output device, such as a monitor.

### BRIEF DESCRIPTION OF THE DRAWINGS

The present invention will now be discussed, by way of example only with reference to certain embodiments and the following Figures, in which:

FIG. 1 is a schematic representation of an imaging system in accordance with an embodiment of the invention;

FIG. 2 is a block diagram of the processing unit shown in FIG. 1;

FIG. 3 is a schematic representation of the monitoring unit shown in FIG. 1; and,

FIG. 4 is a block diagram of the monitoring unit shown in FIG. 3;

### DETAILED DESCRIPTION OF THE INVENTION:

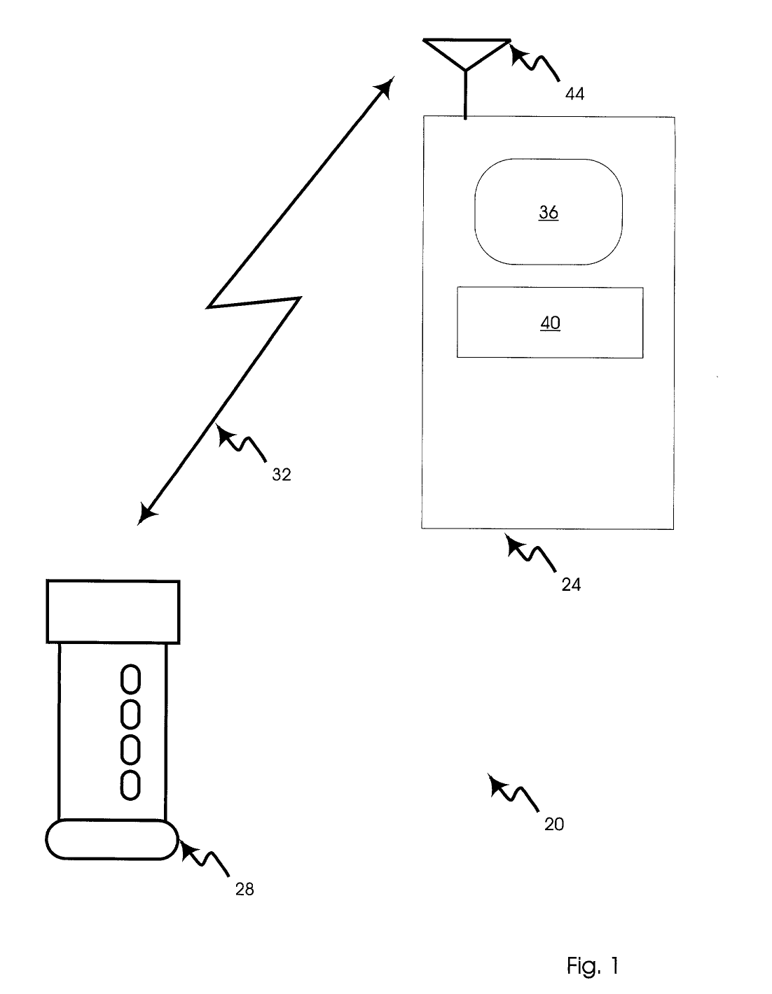

Referring now to FIG. 1, an imaging system in accordance with an embodiment of the invention is indicated generally at 20. In a present embodiment, system 20 is an ultrasound imaging system, comprising a processing unit 24 and a hand-held mobile unit 28 which are interconnected via a wireless link 32.

Processing unit 24 includes a monitor 36 but can also include an audio speaker or any other type of output device as will occur to those of skill in the art. Processing unit 24 also includes a keyboard 40, but can also include other types of input devices such as a trackball. Processing unit further includes an antenna 44 for receiving and transmitting communications over link 32.

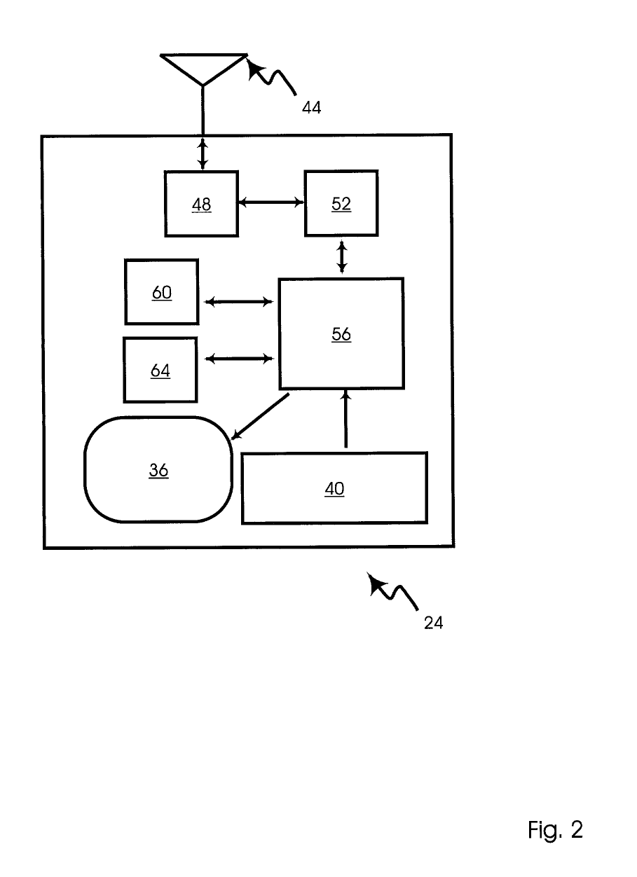

Referring now to FIG. 2, a block diagram shows processing unit 24 in greater detail. As shown in FIG. 2, antenna 44 connects to a radio 48, which in turn connects a microprocessor 56 through a modem 52. Microprocessor 56 interconnects a random access memory (RAM) unit 60, and a storage unit 64, such as a hardisc drive and/or a floppy drive. Microprocessor is also operably connected to monitor 36 to present useroutput thereon and to keyboard 40 to receive user-input therefrom. Collectively, monitor 36, keyboard 40, microprocessor 56, RAM unit 60, and storage unit 64 cooperate to provide processing unit 24 with substantially the same image processing, storage, and output capabilities of conventional ultrasound processing units. In addition, however, microprocessor 56 is also operably connected to modem 52 and radio 48 in order to be able to receive, over wireless link 32, signaling information about the state of mobile unit 28, and digital data representing images captured by mobile unit 28, the details of which will be explained in greater detail below. Further, microprocessor 56 is also operably connected to modem 52 and radio 48 in order to be able to transmit, over wireless link 32, signaling information that informs mobile unit 28 of the state of processing unit 24.

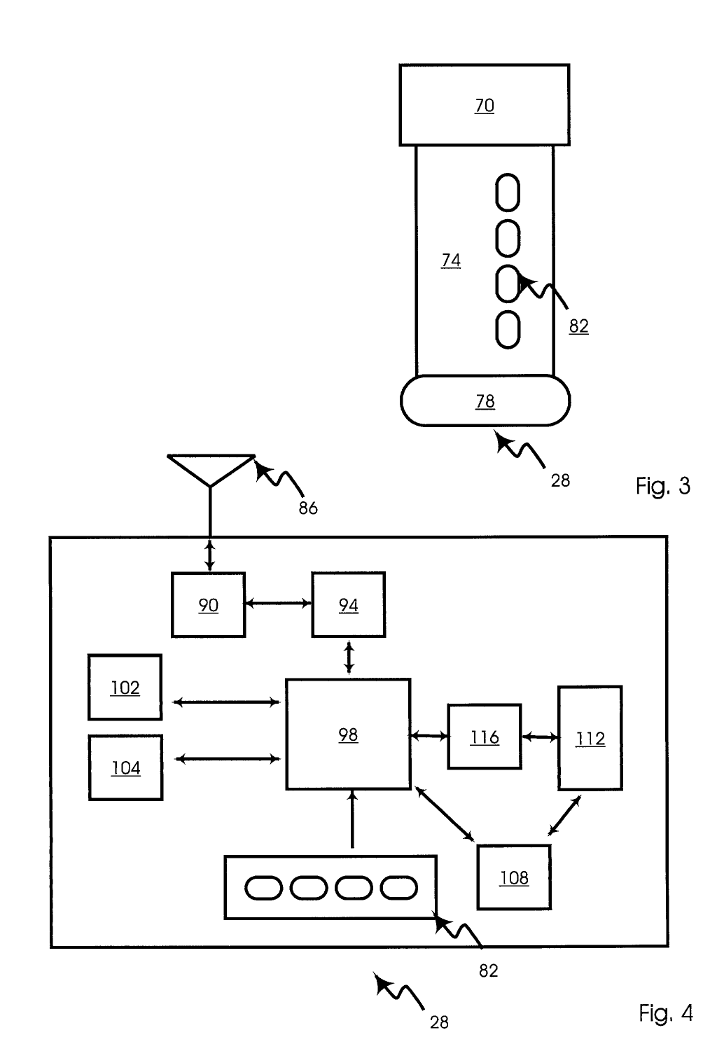

Referring now to FIGS. 3 and 4, mobile unit 28 is shown greater detail. As seen in FIG. 3, in a present embodiment the exterior housing of mobile unit 28 is generally cylindrical and characterized by a radio portion 70, an intermediate hand-grip 74, and a transducer portion 78. Radio portion houses the radio equipment required to allow mobile unit 28 to communicate with processing unit 24 via link 32. Hand-grip 74 is preferably shaped and sized so as to be comfortably grasped by the hand of an operator. In a present embodiment, hand-grip 74 is also characterized by a plurality of keys 82, which are operable to allow the operator to provide input instructions to mobile unit 28 and processing unit 24 via link 32. While not included in the present embodiment, mobile unit 28 can optionally include a series of light-emitting diodes and/or a speaker to provide an operator with status or other output information relevant to the operator.

Referring now to FIG. 4, a block diagram shows mobile unit 28 in greater detail. As shown in FIG. 4, mobile unit 28 includes an antenna 86, a radio 90 and a modem 94 (all of which are typically housed within radio portion 70). Modem 94 connects to a microprocessor 98, which interconnects a random access memory (RAM) unit 102, and a storage unit 104, such as an EEPROM or a smart-card. Microprocessor 102 is also operably connected to keys 82 in order to receive user-input therefrom. Microprocessor 102 is also connected to a portable power-supply 108, such as a rechargeable lithium battery or the like. (Collectively, microprocessor 98, RAM unit 102, storage unit 104 and power-supply 108 are typically housed within hand-grip 74).

Mobile unit 28 also includes transducer 112 connects to microprocessor 98 through an analog-to-digital (A/D) converter 116. (Collectively, transducer 112 and A/D converter 116 are typically housed within transducer portion 78). Transducer 112 receives power from power-supply 108. Transducer 112 can be any suitable or desirable type of ultrasound transducer, which can be chosen and/or configured according to the type of ultrasound imaging operation preferred by the operator. A discussion of exemplary design parameters for transducer 112 are found in Chapter 20 of Christensen 's Physics of Diagnostic Radiology, 4^{th} ed., Curry et al, Lea & Febiger, Philadelphia, 1990. Other suitable design parameters and/or configurations for transducer 112 will occur to those of skill in the art, such as the color transducers available in the ECCOCEE™ CX Ultrasonic Diagnostic System available from Toshiba America Medical Systems, Inc.

Collectively A/D converter 116 and transducer 112 can cooperate to receive ultrasonic images from patients and receive those gathered images into digital format for compression by microprocessor 98 (using compression algorithms stored on storage unit 104). Having compressed the digital images, they are then modulated by modem 94 and transmitted over link 32 via radio 90 and antenna 86.

In operation, an operator will commence the gathering of images by turning on both processing unit 24 and mobile unit 28. While processing unit 24 receives its power from a wall-outlet or other fixed power source, mobile unit 28 will typically be stored within a recharging station so that, when it is needed and powered on, the power-supply 108 therein is fully charged and available for powering mobile unit 28.

Having powered on mobile unit 28 and processing unit 24, signaling information is passed therebetween over link 32. Such signaling information typically includes a handshaking protocol; for example, mobile unit 28 may search for a synchronization signal being continuously generated by processing unit 24, and having found such a synchronization signal, will respond with an acknowledgement signal. At this point, monitor 36 may output a ‘ready’ message, indicating that mobile unit 28 and processing unit 24 have established communication.

Next, the operator places processing unit 24 over the area of the patient to be examined. For example, where the patient is a pregnant women, the processing unit 24 can be gripped by the operator and transducer portion 78 oriented over the patient's abdomen in the usual manner. The operator can then use keys 82 to activate transducer 112, thereby causing transducer 112 to capture an ultrasound image of the patient's fetus. Next, the captured image is converted into a digital image by A/D converter 116, where it is then passed to microprocessor 98 for compression, and then to modem 94 for modulation and then transmitted over link 32 via radio 90 and antenna 86.

The transmitted image data is than received by radio 48 via antenna 44 of processing unit 24, which then passes the transmitted image data to modem 52 for demodulation. Next, the demodulated image data is passed to microprocessor 56 which decompresses the image data, and presents the captured image on monitor 36.

The foregoing operation can then be repeated any number of times to capture a sufficient number of images of the patient's fetus. Furthermore, each received image can be time and date-stamped and stored on storage unit 64 for subsequent retrieval and review.

While the foregoing discussion covers certain exemplary embodiments of the invention, it will be understood that there are variations, subsets and alterations that may be affected thereto and which are within the scope of the invention. For example, mobile unit 28 can be modified to continuously capture and transmit ultrasound images to processing unit 24 for display thereon and subsequent storage.

Further, the configuration of mobile unit 28 is not particularly limited. For example, the shape of mobile unit 28 need not be cylindrical, but be of any shape that is comfortable for grasping. In addition, the battery within mobile unit 28 can be removable and/or rechargeable, as desired.

The configuration of processing unit 24 can also be varied. For example, monitor 36 could be a flat-screen device detachable from processing unit 24 and operable to communicate wirelessly therewith, so that monitor 36 can be situated near the patient in an ergonomic manner in relation to the operator of mobile unit 28.

In addition mobile unit 28 and/or processing unit 24 can be equipped with a microphone and appropriate audio processing equipment to record annotations about each captured image or stream of images. Such annotations can be stored in conjunction with each image on storage unit 64 to accompany any subsequent review or study of stored images.

While the embodiments discussed herein refer to communications carried by radio signals over a wireless link, other types of wireless signals are within the scope of the invention, such as ultrasound, optical, or infrared. Other types of imaging techniques are also contemplated, such as Nuclear medicine cameras, CT cameras, and MR cameras, and angiography equipment or any medical imaging equipment composed of multiple parts where an imaging device links a camera or transducer to a data processing/recording station linked by cables.

It is also contemplated that the device and system included herein could include voice recognition equipment that would allow the control over the equipment by voice commands. For example, instead of using keys on mobile unit 28, a microphone and voice recognition software could be used to receive commands that indicate when it is desired to capture a particular image.

The present invention provides a novel medical imaging device and system that includes a processing unit and a mobile unit which are interconnected by a wireless link. In one embodiment the mobile unit is operable to capture ultrasonic images from a patient, which are transmitted over the wireless link to the mobile unit for display and/or storage. The wireless link allows the operator to move freely throughout the operating arena, without being tangled within cords and allowing the patient to remain relatively undisturbed while simultaneously allowing the operator to have full access to the entire patient's body.

The above-described embodiments of the invention are intended to be examples of the present invention and alterations and modifications may be effected thereto, by those of skill in the art, without departing from the scope of the invention which is defined solely by the claims appended hereto.

We claim:

1. A system for gathering images comprising:
a mobile unit having a transducer, a microprocessor, a modem and a radio for gathering images and transmitting said images over a wireless link; and
a processing unit having a radio, a modem, a microprocessor and an output device for receiving, via said wireless link, said transmitted images and presenting said images on said output device.

2. The system according to claim 1 wherein said transducer is an ultrasound transducer.

3. The system according to claim 1 wherein said mobile unit further includes a keypad for receiving user input representative of desired instructions for controlling said system.

4. The system according to claim 1 wherein said output device is a monitor.

5. The system according to claim 1 wherein said processing unit further includes a storage device for maintaining copies of said transmitted images for further review.

6. The system according to claim 5 wherein said storage device includes removable media.

7. The system according to claim 5 wherein at least one of said mobile unit and said processing unit include a microphone for receiving annotations, and said storage device is operable to maintain copies of said annotations.

8. The system according to claim 7 wherein said stored annotations are indexed in relation to a respective stored image.

9. A method for gathering images comprising:
capturing an analog image representation of at least a portion of a patient;
converting said analog image representation into a digital representation; and,
transmitting said image over a wireless link.

10. A method for gathering images comprising:
capturing an analog image representation of at least a portion of a patient;
converting said analog image representation into a digital representation; and,
compressing said image;
modulating said image into a format for wireless transmission;
transmitting said modulated image over a wireless link.
receiving said transmitted image at a processing unit;
demodulating said received image; and,
presenting said image on an output device.

11. The method according to claim 10 image is captured by an ultrasonic transducer.

12. The method according to claim 10 wherein said output device is a monitor.

13. The method according to claim 10 further comprising the step of storing, on a storage device, a copy of said image for subsequent retrieval.

14. The method according to claim 13 further comprising the step of receiving annotations and storing said annotations for subsequent retrieval.

15. The method according to claim 15 wherein said stored annotations are indexed in relation to a respective stored image.
